(19) United States
(12) Patent Application Publication
(10) Pub. No.: US 2021/0330211 A1
(43) Pub. Date: Oct. 28, 2021

(54) EXERCISE MANAGEMENT METHOD AND SYSTEM USING ELECTROMYOGRAPHY SENSOR

(71) Applicant: HHS CO.,LTD., Ulsan (KR)

(72) Inventors: Hyung Seob HAN, Ulsan (KR); Kyoung Young SONG, Ulsan (KR); Oh Guk KWON, Ulsan (KR); Tae Young KIM, Ulsan (KR)

(21) Appl. No.: 16/317,525

(22) PCT Filed: Jun. 29, 2017
(86) PCT No.: PCT/KR2017/006897

§ 371 Date: Nov. 5, 2020

(30) Foreign Application Priority Data
Jul. 12, 2016 (KR) 10-2016-0088023

(51) Int. Cl. A61B 5/0488; A63B 24/00; A61B 5/04; A61B 5/00; G16H 40/67; G16H 20/30; G16H 50/30; G09B 5/02

(52) U.S. Cl. CPC A61B 5/0488; A63B 24/0062; A61B 5/04012; A61B 5/7246; H04L 67/12; G16H 20/30; G16H 50/30; G09B 5/02; A61B 5/0022; G16H 40/67

(57) ABSTRACT

Disclosed is an exercise guidance system using an electromyography sensor, the system including: a control server receiving exercise information by working in conjunction with a monitoring module, in which an exercise guidance application is installed, over a wired/wireless communication network, the control server providing analysis information on user's exercise; and a signal processing module receiving detection signals from multiple electromyography sensors attached on a user body, calculating muscle activity by analyzing the detection signals, and providing a result of the calculation to the monitoring module. According to the embodiment, the cost burden of personal training is reduced, and the monotony of exercising along is reduced. Also, there is no limitation of place and time because exercise is possible anywhere. Also, the electromyography sensor works in conjunction with the smartphone to provide visualization of the user's exercise volume, the user's muscles, and the like, thereby facilitating efficient exercising.

### CROSS REFERENCE TO RELATED APPLICATION

The present application claims priority to Korean Patent Application No. 10-2016-0088023, filed Jul. 12, 2016, the entire contents of which is incorporated herein for all purposes by this reference.

### BACKGROUND OF THE INVENTION

### Field of the Invention

The present invention relates to an exercise guidance method and system using an electromyography sensor. More particularly, the present invention relates to an exercise guidance method and system using a wearable electromyography sensor.

### Description of the Related Art

Recently, as scientific technology has advanced, a living environment has become enriched and convenient. However, due to lack of physical activity and exercise, chronic adult diseases, such as hypertension, diabetes, cardiovascular disease, chronic fatigue, and the like are problems.

Therefore, as interest in health and awareness of the need for exercise have increased, many people exercise or plan for exercise.

However, in order to get a program for exercise suitable for oneself, it is necessary to visit hospital or professional health center to receive guidance, such as personal training, thus the process is time consuming and costly.

In the meantime, with the development of information and communication technology and popularization of smartphones, an environment is created wherein various types of information are transmitted and received without limitation of physical time and space.

Accordingly, there has been an attempt to reduce the cost burden of personal training and to reduce the monotony of exercise so as to provide more logical and systematic exercise methods.

In Korean Patent Application Publication No. 10-2014-0113125, a technique for providing a custom-made individual health service method to a mobile terminal is disclosed.

However, it is impossible to measure the exercise volume and efficiency of an individual, and to provide objective feedback, resulting in side effects, such as injury, decrease in exercise ability, and the like.

The foregoing is intended merely to aid in the understanding of the background of the present invention, and is not intended to mean that the present invention falls within the purview of the related art that is already known to those skilled in the art.

### SUMMARY OF THE INVENTION

Accordingly, the present invention has been made keeping in mind the above problems occurring in the related art, and the present invention is intended to propose an exercise guidance method and system using a wearable electromyography sensor.

In order to achieve the above object, according to one aspect of the present invention, there is provided an exercise guidance system using an electromyography sensor, the system including: a control server receiving exercise information by working in conjunction with a monitoring module, in which an exercise guidance application is installed, over a wired/wireless communication network, the control server providing analysis information on a user's exercise; and a signal processing module receiving detection signals from the multiple electromyography sensors attached on a user body, calculating muscle activity by analyzing the detection signals, and providing a result of the calculation to the monitoring module.

The signal processing module may include: a signal analysis unit analyzing the detection signals and selecting both an intrinsic mode function (IMF) equal to or larger than a threshold value and a subband with a maximum rate of change; and a feature extraction unit calculating the muscle activity from the IMF and the subband with the maximum rate of change.

The muscle activity may be calculated using muscular contraction tonus, muscle fatigue, and muscular contraction timing.

The muscular contraction tonus may be calculated from RMS of the IMF and the subband with the maximum rate of change, the muscle fatigue may be calculated from a median frequency, and the muscular contraction timing may be calculated from a cross-correlation function between the multiple electromyography sensors.

According to another aspect of the present invention, there is provided an exercise guidance method using an electromyography sensor, wherein exercise guidance is performed via the multiple electromyography sensors and an exercise guidance application of a monitoring module, the method including: receiving exercise information from the monitoring module by working in conjunction therewith over a wired/wireless communication network, and receiving attachment position information of the electromyography sensors from the electromyography sensors; receiving detection signals from the electromyography sensors when starting exercise; calculating muscle activity by analyzing the detection signals, and providing a result of the calculation to the monitoring module; and seeking an improvement plan by analyzing the exercise information and the muscle activity, and providing the improvement plan as feedback to the monitoring module.

The calculating of the muscle activity may include: analyzing the detection signals and selecting both an intrinsic mode function (IMF) equal to or larger than a threshold value and a subband with a maximum rate of change; and calculating muscular contraction tonus from RMS of the IMF and the subband with the maximum rate of change, calculating muscle fatigue from a median frequency, and calculating muscular contraction timing from a cross-correlation function between channels so as to be provided as the muscle activity.

According to the embodiment, the cost burden of personal training is reduced, and the monotony of exercising along is reduced.

Also, there is no limitation of place and time because exercise is possible anywhere. Also, the electromyography sensor works in conjunction with a smartphone to provide visualization of the user's exercise volume, the user's muscles, and the like, thereby facilitating efficient exercising.

### BRIEF DESCRIPTION OF THE DRAWINGS

The above and other objects, features and other advantages of the present invention will be more clearly understood from the following detailed description when taken in conjunction with the accompanying drawings, in which:

FIG. 1 is a diagram illustrating a configuration of an entire system that includes an exercise guidance system using an electromyography sensor according to an embodiment of the present invention;

FIG. 2 is a diagram illustrating the entire system of FIG. 1;

FIG. 3 is a diagram illustrating a detailed configuration of an electromyography sensor;

FIG. 4 is a diagram illustrating a detailed configuration of a signal processing module;

FIG. 5 is a flowchart illustrating an operation of the entire system of FIG. 1; and

FIG. 6 is a flowchart illustrating in detail a process of calculating the muscle activity by a signal processing module of FIG. 5.

### DETAILED DESCRIPTION OF THE INVENTION

Hereinbelow, exemplary embodiments of the present invention will be described in detail with reference to the accompanying drawings such that the present invention can be easily embodied by those skilled in the art to which this present invention belongs. However, the present invention may be embodied in various different forms and should not be limited to the embodiments set forth herein. Further, in order to clearly explain the present invention, portions that are not related to the present invention are omitted in the drawings, and like reference numerals designate like elements throughout the specification.

Throughout the specification, when a part is referred to as being “connected” to another part, it includes not only being “directly connected”, but also being “electrically connected” by interposing the other part therebetween.

Throughout the specification, when a part “includes” an element, it is noted that it may further include other elements, but does not exclude other elements, unless specifically stated otherwise. Also, the terms “˜part”, “˜unit”, “module”, and the like mean a unit for processing at least one function or operation and may be implemented by a combination of hardware and/or software.

Hereinafter, exemplary embodiments of the present invention will be described in detail with reference to the accompanying drawings.

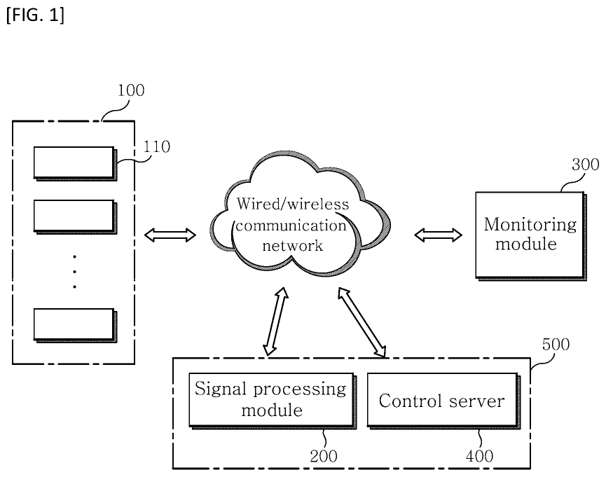

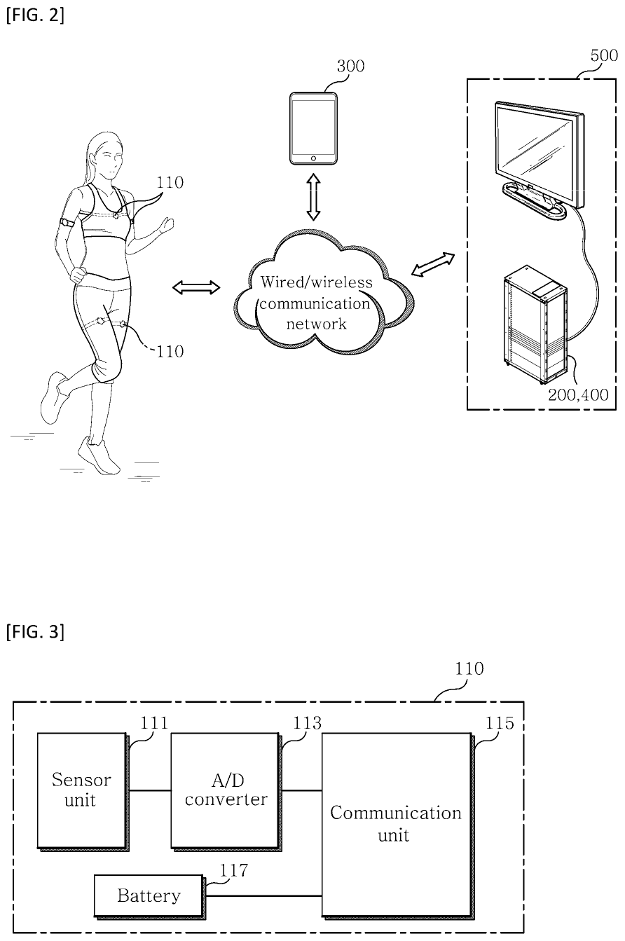

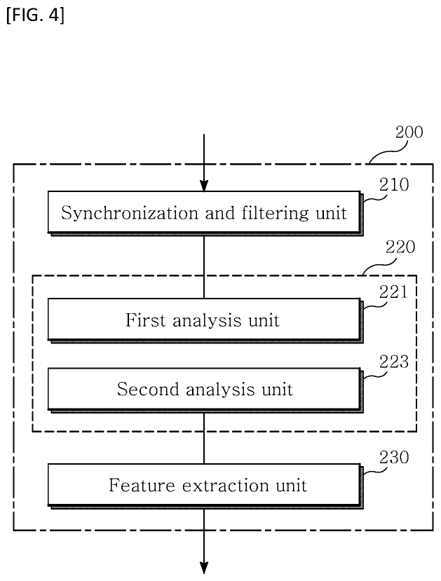

FIG. 1 is a diagram illustrating a configuration of an entire system that includes an exercise guidance system using an electromyography sensor according to an embodiment of the present invention. FIG. 2 is a diagram illustrating the entire system of FIG. 1. FIG. 3 is a diagram illustrating a detailed configuration of an electromyography sensor. FIG. 4 is a diagram illustrating a detailed configuration of a signal processing module.

Referring to FIG. 1, the entire system that includes an exercise guidance system 500 (hereinafter, referred to as “an exercise guidance server 500″) using an electromyography sensor according to the embodiment of the present invention includes: a monitoring module 300; the exercise guidance server 500; an electromyography sensor 100; and exercise equipment (not shown).

The monitoring module 300 is a terminal that a user uses to access the exercise guidance server 500 and downloads an exercise guidance application from the exercise guidance server 100 for installation. Examples of the terminal include a smartphone, a notebook, a tablet PC, or the like that is provided with a display window.

The monitoring module 300 works in conjunction with the exercise guidance server 500 by the wired/wireless Internet. Here, the wireless Internet may be WiFi, Bluetooth, or the like.

The monitoring module 300 may install the exercise guidance application with respect to the exercise guidance server 500, may run the application to transmit various types of information to the exercise guidance server 500, and may receive various types of information from the exercise guidance server 500.

The electromyography sensor 100 includes multiple sensor modules 110, and each sensor module 110 is realized as a wearable device.

That is, each electromyography sensor module 110 is formed in a band-type structure in such a manner as to be directly attached on a user body.

As the user exercises, the electromyography sensor module 110 may perform electromyography with respect to the movement and may transmit a detection signal.

The electromyography sensor module 110 is provided with the communication unit 115 for wireless communication with the signal processing module 200 in such a manner as to transmit the detection signal generated according to the movement of the user to the signal processing module 200.

The multiple sensor modules 110 of the electromyography sensor 100 are attached on different portions of the user body and simultaneously transmit respective detection signals.

That is, as shown in FIG. 2, it may be attached to the user's arms, legs, chest, buttocks, and the like without any limitation. It may be attached at the position of the muscle targeted when the user exercises so as to detect the exercise effect on the target muscle.

Each electromyography sensor module 110 has a unique serial number that is transmitted with the generated detection signal to the signal processing module 200, so that the signal processing module 200 identifies each sensor module 110.

Each electromyography sensor module 110 may have a detailed configuration as shown in FIG. 3.

Referring to FIG. 3, each electromyography sensor module 110 may include a sensor unit 111, an A/D converter 113, a communication unit 115, and a battery 117.

The sensor unit 111 is an electromyography sensor that detects vital signals accompanied by the activity of the muscles detected by electrodes which are attached on the muscles so as to perform surface electromyography. The electromyography sensor measures the amount of the voltage and the current flowing around the muscle, and the frequency by attaching two electrodes, a reference electrode and a measurement electrode, on the user body.

Here, the potential difference between the two electrodes is amplified by an amplifier of the sensor, and a filter removes the power noise of 60 Hz. Further, a low-pass filter removes the high-frequency noise, thereby detecting an electromyography signal.

The A/D converter 113 digitizes the electromyography signal from the sensor unit 111 for output. The communication unit 115 transmits the digital signal to the signal processing module over the wired/wireless communication network. Here, the communication unit 115 transmits the serial number of each electromyography sensor together.

Further, the electromyography sensor module 110 includes the battery 117. The battery 117 may be a rechargeable battery 117.

In the meantime, as shown in FIG. 1, the exercise guidance server 500 may include the signal processing module 200 and a control server 400. The signal processing module 200 and the control server 400 may be physically separated from each other, or may be separated from each other within one PC in a functional manner.

The signal processing module 200 receives various detection signals from the electromyography sensor 100 over a wired/wireless communication network, and performs signal processing and reading on the resulting signals so as to calculate muscle activity which is a valid feature value.

More specifically, referring to FIG. 4, the signal processing module 200 may include a synchronization and filtering unit 210, a signal analysis unit 220, and a feature extraction unit 230.

The synchronization and filtering unit 210 synchronizes, for each channel, multiple detection signals received from respective electromyography sensor modules 110 and performs noise filtering.

The signal analysis unit 220 may include a first analysis unit 221 and a second analysis unit 223 that obtain a valid feature value from the detection signal.

The first analysis unit 221 breaks down the filtered detection signal into multiple intrinsic mode functions (IMF) by using empirical mode decomposition (EMD), and obtains a spectrum value for each IMF to obtain a value of IMFs equal to or larger than a threshold value from the harmonic characteristics and the power ratio.

The second analysis unit 223 breaks down the filtered detection signal into multiple subbands by using a discrete wavelet transform (DWT), obtains the average, variance, skewness, and kurtosis of each band, and selects the subband with the maximum rate of change, wherein the subband has the largest rate of change among the rates of change of values obtained in respective subbands for each frame.

As described above, the value of IMFs and the subband with the maximum rate of change are defined as valid feature values.

In the meantime, the feature extraction unit 230 calculates the muscle activity from the selected valid feature values. Specifically, the RMS is obtained from the selected IMFs and the selected subband so as to calculate muscular contraction tonus, and the muscle fatigue is calculated from the median frequency. Further, the feature extraction unit 230 analyzes the muscular contraction timing using a cross-correlation function between channels.

As described above, the feature extraction unit 230 may extract and transmit the muscular contraction tonus, fatigue, and muscular contraction timing as the muscle activity.

In the meantime, the control server 400 may check over the wired/wireless communication network whether the user is an exercise guidance service subscriber, and may receive body information and exercise information of the exercise guidance service subscriber when the user is the exercise guidance service subscriber. The information may be analyzed to propose a customized exercise program and to provide an improvement plan for the current exercise method.

Further, through an archive analysis, exercise information on various subscribers is accumulated for storage and analyzed by time, age, gender, and region so as to seek user's favorite exercise devices, time-based exercise habits, exercise trends by region, a problem with exercise for all rather than individuals, and an improvement plan.

The exercise guidance server 500, which includes the signal processing module 200 and the control server 400, provides the exercise guidance application that is installed in the monitoring module 300 to display the improvement plan and feedback for exercise and to transmit start information, and the like to each electromyography sensor module 110.

The exercise guidance system 500 operates when the user installs the exercise guidance application in the monitoring module 300, for example, the user's smartphone and attaches the multiple electromyography sensor modules 110 on body portions to exercise.

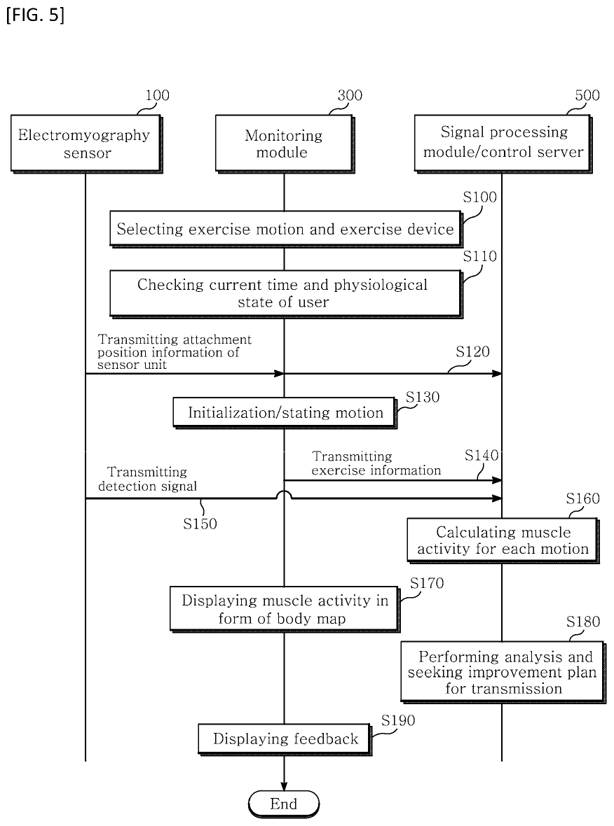

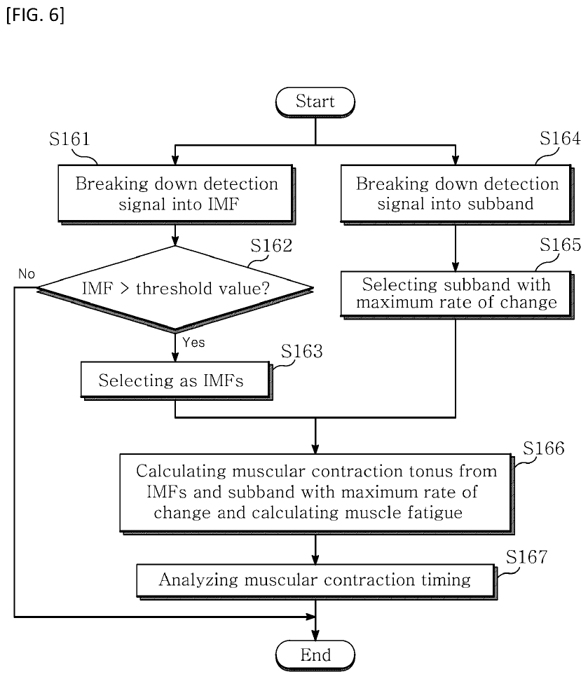

Hereinafter, an operation of the exercise guidance system according to the embodiment of the present invention will be described with reference to FIGS. 5 and 6.

FIG. 5 is a flowchart illustrating an operation of the entire system of FIG. 1. FIG. 6 is a flowchart illustrating in detail a process of calculating the muscle activity by a signal processing module of FIG. 5.

First, the user selects the exercise motion while holding the monitoring module 300, for example, the smartphone, in which the exercise guidance application is installed, and selects the exercise device when the exercise devices to be used are present at step S100. The selection of the exercise device may be omitted, when the device is not required.

Next, the user starts the exercise guidance application of the smartphone, and inputs the current exercise time and the physiological state of the user who exercises at step S110. The physiological state may be gender, height, weight, age, abdominal obesity, and the like. The information on the physiological state may be obtained by various types of measurement devices, for example, a scale, a tapeline, InBody, and the like.

Further, the body information may be transmitted to the exercise guidance server 500 over the wired/wireless communication network.

Next, the exercise guidance server 500 makes a request to the electromyography sensor 100 for attachment position information of each sensor unit 111 of the sensor module 110, and receives the position information at step S120. Here, the position information is also transmitted to the monitoring module 300.

When the monitoring module 300 receives the position information, the device is initialized and the user starts exercise at step S130.

Here, the monitoring module 300 may transmit corresponding exercise information, namely, information on time, device, physiological state, and the like to the exercise guidance server 500 via the application at step S140.

When starting exercise, the electromyography sensor 100 generates and transmits the detection signal to the signal processing module 200 of the exercise guidance server 500 at step S150.

Next, the signal processing module 200 calculates the muscle activity for each motion from the detection signal and transmits the result to the monitoring module 300 at step S160.

The process of calculating the muscle activity is shown in FIG. 6.

Specifically, first, the detection signal is received, and the detection signal is broken down into multiple intrinsic mode functions (IMF) by using the empirical mode decomposition (EMD) at step S161.

Next, the spectrum value for each of the IMFs is obtained, and from the harmonic characteristics and the power ratio, the IMFs are selected when being equal to or larger than the threshold value at step S162.

In the meantime, the filtered detection signal is broken down into multiple subbands using the discrete wavelet transform (DWT) at step S164. Next, the average, variance, skewness, and kurtosis of each band are obtained; the subband with the maximum rate of change is selected, wherein the subband has the largest rate of change among the rates of change of values obtained in respective subbands for each frame at step S165.

Here, the value of IMFs and the subband with the maximum rate of change are defined as valid feature values, and the muscle activity is calculated from the valid feature values at step S166. Specifically, the RMS is obtained from the selected IMFs and the selected subband so as to calculate muscular contraction tonus, and the muscle fatigue is calculated from the median frequency.

Next, the muscular contraction timing is analyzed using the cross-correlation function between channels, namely the sensor modules 110 at step S167.

As described above, the muscular contraction tonus, fatigue, and muscular contraction timing are extracted and transmitted to the monitoring module 300 as the muscle activity.

The monitoring module 300 receives and displays the muscle activity at step S170. Here, the activity via the exercise guidance application is displayed in the form of a body map in such a manner as to be easily and effectively perceived by the user.

In the meantime, the control server 400 of the exercise guidance server 500 analyzes the muscle activity from the signal processing module 200 and the exercise information to determine the exercise state of the user, and seeks the improvement plan for the exercise state to transmit the result to the monitoring module 300.

The monitoring module 300 receives the result via the application, displays the result as an exercise program for feedback to the user, and terminates the application.

The control server 400 performs an archive analysis involving the exercise program and updates a database.

As described above, the wearable electromyography sensor is attached on the user's exercise portion, and the muscle activity is read and displayed in real time while exercising, thereby providing the accuracy of the exercise and the improvement plan and enabling the efficient exercise.

Although the present invention has been described with reference to the exemplary embodiments, those skilled in the art will appreciate that various modifications and variations can be made in the present invention without departing from the spirit or scope of the invention described in the appended claims.

What is claimed is:

1. An exercise guidance system using an electromyography sensor, the system comprising:
a control server receiving exercise information by working in conjunction with a monitoring module, in which an exercise guidance application is installed, over a wired/wireless communication network, the control server providing analysis information on a user's exercise; and
a signal processing module receiving detection signals from the multiple electromyography sensors attached on a user body, calculating muscle activity by analyzing the detection signals, and providing a result of the calculation to the monitoring module.

2. The system of claim 1, wherein the signal processing module comprises:
a signal analysis unit analyzing the detection signals and selecting both an intrinsic mode function (IMF) equal to or larger than a threshold value and a subband with a maximum rate of change; and
a feature extraction unit calculating the muscle activity from the IMF and the subband with the maximum rate of change.

3. The system of claim 2, wherein the muscle activity is calculated using muscular contraction tonus, muscle fatigue, and muscular contraction timing.

4. The system of claim 3, wherein the muscular contraction tonus is calculated from RMS of the IMF and the subband with the maximum rate of change,
the muscle fatigue is calculated from a median frequency, and
the muscular contraction timing is calculated from a cross-correlation function between the multiple electromyography sensors.

5. An exercise guidance method using an electromyography sensor, wherein exercise guidance is performed via the multiple electromyography sensors and an exercise guidance application of a monitoring module, the method comprising:
receiving exercise information from the monitoring module by working in conjunction therewith over a wired/wireless communication network, and receiving attachment position information of the electromyography sensors from the electromyography sensors;
receiving detection signals from the electromyography sensors when starting exercise;
calculating muscle activity by analyzing the detection signals, and providing a result of the calculation to the monitoring module; and
seeking an improvement plan by analyzing the exercise information and the muscle activity, and providing the improvement plan as feedback to the monitoring module.

6. The method of claim 5, wherein the calculating of the muscle activity comprises:
analyzing the detection signals and selecting both an intrinsic mode function (IMF) equal to or larger than a threshold value and a subband with a maximum rate of change; and
calculating muscular contraction tonus from RMS of the IMF and the subband with the maximum rate of change, calculating muscle fatigue from a median frequency, and calculating muscular contraction timing from a cross-correlation function between channels so as to be provided as the muscle activity.
